(19) United States
(12) Patent Application Publication
(10) Pub. No.: US 2007/0269792 A1
(43) Pub. Date: Nov. 22, 2007

(54) Cross-linked collagen matrix for producing a skin equivalent

(76) Inventors: August Bernd, Bingen-Kempten (DE); Wolfgang Friess, Iffeldorf (DE); Karsten Rudiger Newes, Mainz (DE); Martina Spiegel, Oberursel (DE); Henriette Bendz, Munchen (DE); Stefan Kippenberger, Frankfurt am Main (DE)

Correspondence Address: PAUL & PAUL, 2000 MARKET STREET, PHILADELPHIA, PA, 19103-3229, US

(21) Appl. No.: 11/705,674

(22) Filed: Feb. 13, 2007

Related U.S. Application Data
(63) Continuation of application No. PCT/EP05/08478, filed on Aug. 5, 2005.

(30) Foreign Application Priority Data
Aug. 13, 2004 (DE) 10 2004 039 537.3

(51) Int. Cl. A61K 35/12; C12Q 1/00

(52) U.S. Cl. 435/1.1; 424/572; 435/4

(57) ABSTRACT

A whole skin model is made by a process for the production of a whole skin model comprising the steps of: (a) providing a poorly soluble collagen obtained from collagen-containing tissue; (b) forming a homogeneous aqueous suspension by mixing the collagen with an aqueous medium; (c) forming a first matrix (A) by lyophilizing the collagen suspension; (d) forming a second matrix (B) by cross-linking the collagen in the first matrix (A) to form a mechanically stabilized second matrix (B); (e) sowing fibroblasts onto the second matrix (B) and allowing the fibroblasts to grow; (f) sowing keratinocytes onto the second matrix B and allowing the keratinocytes to grow; (g) further cultivating the fibroblasts and keratinocytes growing in and on matrix (B) to form a complete whole skin model comprised of a dermal and epidermal part.

### CROSS-REFERENCE TO RELATED APPLICATIONS

This application is a continuation under 35 U.S.C. § 365(c) and 35 U.S.C. § 120 of International Application No. PCT/EP2005/008478, filed on Aug. 5, 2005. This application also claims priority under 35 U.S.C. § 119 of German Application No. DE 10 2004 039 537.3, filed on Aug. 13, 2004. Each application is incorporated herein by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

This invention relates to a cross-linked collagen matrix for producing a skin equivalent, a dermis equivalent, an epidermis equivalent and a skin equivalent based on such a collagen matrix and to processes for producing the collagen matrix and the dermis, epidermis or skin equivalent.

Skin-typical whole skin models, which may also be referred to as in-vitro skin equivalents, may be used as test skin in dermatology and in allergology for testing substances, for example, potential medicaments or cosmetics, or agents, such as light and heat, for their pharmacological or cosmetic effects, more particularly irritation, toxicity and inflammation effects, and for their compatibility. In addition, such a system can be used for various immunological, histological and molecular-biological tasks. These include, for example, studies on wound healing and studies on the penetration and absorption of substances. The testing of substances using such whole skin models affords major advantages over animal tests and tests with human volunteers because the results obtained with whole skin models are more reproducible and the tests can be carried out more quickly and at less cost.

(2) Description of Related Art, Including Information Disclosed Under 37 C.F.R. §§ 1.97 and 1.98

In recent years, human cell cultures have generally been used as in-vitro systems for testing raw materials and products. Organ-like human tissues or bioartificial constructs and co-culture systems represent a further development of the cell culture technique. The results obtained can be applied to human beings even better than the results obtained with single cell cultures.

Published European Application EP 0 197 090 B1 discloses a process for forming a skin equivalent in which a hydrated collagen lattice is produced by mixing an acidic collagen solution with contractile cells, for example, fibroblasts. After neutralization of the pH, collagen fibrils are precipitated in the collagen lattice. The contractile cells attach themselves to the collagen lattice and cause it to contract, resulting in the formation of a dermis equivalent. By introducing punch skin biopsies into the collagen lattice, keratinocytes from the punch biopsies are able to grow on the surface of the dermis equivalent, resulting in the formation of a skin equivalent.

Published European Application EP 0 285 474 B1 discloses a skin equivalent comprising a dermis equivalent obtained from collagen and fibroblasts and a multilayer epidermis equivalent. The dermis equivalent is inoculated with a human or animal explantate, for example, a hair follicle, to obtain the epidermis equivalent.

Published European Application EP 0 020 753 B1 describes a process for forming tissue, more particularly skin tissue, in which fibroblasts are again introduced into a hydrated collagen lattice and a tissue is formed after contraction of the collagen lattice. Keratinocytes cultivated in vitro beforehand or keratinocytes isolated from foreskin can be applied to that tissue, resulting in the formation of a skin substitute.

Published European Application EP 0 418 035 B1 describes a tissue equivalent comprising a hydrated collagen lattice, which is contracted by a contractile agent, such as fibroblasts, and a collagen gel, which is in contact with a permeable element. The mixture of collagen and contractile agent is applied to the collagen gel, the radial or lateral contraction of the collagen lattice being suppressed by the contact between collagen gel and permeable element, for example, a polycarbonate membrane, so that the lattice only contracts in its thickness. After formation of the dermis equivalent, keratinocytes can then be sown, resulting in the formation of a skin equivalent.

In addition, U.S. Pat. No. 5,861,153 discloses a skin equivalent consisting of an epidermis equivalent on a carrier, the epidermis equivalent comprising keratinocytes and induced or non-induced precursors of Langerhans cells. The carrier may be a fibroblast-containing collagen lattice or dermis sections freed from the epidermis, artificial membranes, a collagen-based subcutaneous substitute or synthetic materials.

U.S. Pat. No. 4,963,489 describes a stroma tissue produced in situ, the stroma cells, for example fibroblasts, enveloping a basic framework which consists of a biologically compatible material, for example, cellulose. The described system may be used inter alia for producing a three-dimensional skin culture system, keratinocytes and melanocytes being applied to the dermis equivalent, i.e. the three-dimensional carrier matrix.

U.S. Pat. No. 5,755,814 describes a skin model system which may be used both as an in-vitro test system and for therapeutic purposes. The system comprises a three-dimensional cross-linked matrix of insoluble collagen with fibroblasts contained therein and stratified layers of differentiated epidermis cells, an epidermis cell layer being in direct contact with the surface of the collagen matrix. The matrix may be cross-linked both by heat treatment and removal of water and by chemical agents, for example, carbodiimide.

U.S. Pat. No. 5,882,248 describes a process for determining the effect of chemical substances or agents on the human skin model system according to U.S. Pat. No. 5,755,814. The interaction between the skin model system and the substances to be tested is determined from the release of substances by cells of the skin model system and the effects on metabolism, proliferation, differentiation and reorganization of those cells.

In addition, International Patent Application No. WO 95/10600 describes a process by which an epidermis equivalent can be obtained. This epidermis equivalent can be used for pharmaceutical and/or cosmetic suntan tests.

International Patent Application No. WO 01/092477 discloses a process for differentiating and/or proliferating isolated dermal fibroblasts, the fibroblasts being cultivated in a three-dimensional gel-like matrix where they are able to proliferate. Besides the fibroblasts to be cultivated, this matrix contains a framework of human or animal collagen formed from a collagen solution, i.e. tissue-typical matrix proteins. The process is said to provide an organoid in-vitro skin model which is made up of two tissue-typical layers, namely a dermis equivalent and an epidermis equivalent. The organotypical skin model is said to correspond largely to native skin both histologically and functionally.

A disadvantage of known skin models is that they generally consist solely of one or more epidermal layer(s) of keratinocytes. In cases where a stratified epidermis is obtained, tissue explantates are often used, which involves the risk of contamination with pathogens, leading to false results where the skin equivalent is subsequently used as test skin. If the described skin models have a dermal part, it often consists of spongy, cross-linked material which, besides collagen, can also contain other non-skin-typical materials. If the dermal part of the skin equivalents described in the prior art consists solely of collagen and fibroblasts, it is subjected to an undefined shrinkage process which is attributable to heavy shrinkage of the collagen gel and the egress of fluid therefrom. The effect of this is that the skin equivalents described in the prior art are only suitable to a limited extent as test skin of a particular size and the results obtained with them can only be applied to a limited extent to native human skin. In addition, conventional skin equivalents with a dermal part of collagen and fibroblasts are complicated to produce because a collagen gel that is difficult to process is used. The gel solution has to be stirred at low temperature, i.e. best on ice, and also has to be pipetted cold because only in this way does it remain liquid. The gel solution is then heated in an incubator, for example, to 37° C. Only under these conditions and at the correct pH does gelation occur. The warmer the gel solution, the more difficult it is to process. In addition, if the gel is heated too strongly, the collagen is in danger of denaturing so that the gel becomes unusable.

Finally, it remains to be pointed out that the skin models known from the prior art are suboptimal in their similarity to skin. In particular, the expression of the important dermal differentiation marker, elastin, is nowhere to be seen in the known skin models in contrast to natural dermis.

Accordingly, the technical problem addressed by the present invention was to provide a human in-vitro whole skin model substantially corresponding to native human skin, which would have both an epidermis layer and a dermis layer, would avoid the above-mentioned disadvantages of the prior art and could be used as test skin, for example, for studying pharmacological and cosmetic effects and for testing dermatological compatibility, and processes and means for producing the in-vitro whole skin model.

### BRIEF SUMMARY OF THE INVENTION

The present invention pertains to a process for the production of a whole skin model comprising the steps of: (a) providing a poorly soluble collagen obtained from collagen-containing tissue; (b) forming a homogeneous aqueous suspension by mixing the collagen with an aqueous medium; (c) forming a first matrix (A) by lyophilizing the collagen suspension; (d) forming a second matrix (B) by cross-linking the collagen in the first matrix (A) to form a mechanically stabilized second matrix (B); (e) sowing fibroblasts onto the second matrix (B) and allowing the fibroblasts to grow; (f) sowing keratinocytes onto the second matrix B and allowing the keratinocytes to grow; (g) further cultivating the fibroblasts and keratinocytes growing in and on matrix (B) to form a complete whole skin model comprised of a dermal and epidermal part. The present invention also pertains to a whole skin model made by the process according to the invention.

### BRIEF DESCRIPTION OF THE SEVERAL VIEWS OF THE DRAWING(S)

Not Applicable

### DETAILED DESCRIPTION OF THE INVENTION

In complete contrast to the collagen gels used in the prior art, the collagen suspension obtained in step b) can advantageously be stored and pipetted without difficulty at room temperature.

The whole skin model obtainable in accordance with the invention is easy to handle by comparison with known models because it is particularly robust. In addition, the whole skin model obtainable in accordance with the invention adheres well to the bottom of the culture vessels so that the as yet incompletely formed skin model does not become prematurely detached and, hence, unusable.

In the context of the present invention, the term “cultivation” means the preferably in-vitro maintenance of the life function of cells, for example, fibroblasts or keratinocytes, in a suitable environment, for example, through the introduction and removal of metabolism educts and products, and more particularly proliferation of the cells.

In the context of the present invention, fibroblasts are understood to be naturally occurring fibroblasts, more particularly fibroblasts occurring in the dermis, genetically modified fibroblasts or fibroblasts emanating from spontaneous mutations or precursors thereof. The fibroblasts may be of animal or human origin.

“Poorly soluble collagen” in the context of the invention is understood to be coarse-fiber collagen which shows no visible swelling or only slight swelling and little or no gelling, above all no gelling, after several hours in aqueous medium. Poorly soluble collagen is preferably obtained from the tendons of animals, more particularly mammals, preferably horses, pigs or cattle, and more preferably from the Achilles tendons or the skin of cattle, more particularly from the Achilles tendons of cattle.

The conversion into a homogeneous collagen suspension can be carried out with, or in, any mixing unit considered suitable by the expert, preferably in a static mixer or with an Ultra Turrax.

The collagen suspension is introduced into receptacles of which the dimensions correspond to those of the required matrixes, for example, into the wells of a microtiter plate.

Before the receptacles, for example, the wells of a microtiter plate, are filled with the collagen suspension, they may be coated with various agents in order subsequently to improve the adhesion of the freeze-dried collagen matrix to the receptacle wall. Suitable coating agents are, for example, collagen, gelatine, polylysine, fibrin or fibrinogen/thrombin or fibronectin. For coating, the inner receptacle walls are first wetted with solutions or suspensions of the agents, the receptacles are dried, so that a layer of the agents is formed on the inner receptacle surface, and, finally, the receptacles are filled with the collagen suspension.

The lyophilization (freeze drying) step c) takes place in these receptacles. If freezing is carried out at a low cooling rate, large ice crystals are obtained and cause differences in concentration and separation in the product. Slow, pronounced ice crystal growth leads to the formation of particularly large ice crystals which, in the presence of a temperature gradient during freezing, are aligned in the direction of the gradient. In this case, the products obtained are permeated by a large-lumen columnar or chimney structure. However, the specific surface of these coarsely grown structures is reduced in size, so that reconstitution behavior can be adversely affected.

For this reason, cooling is normally carried out rapidly in the freeze drying of biological material.

However, applicants have surprisingly found that slow cooling of the collagen suspension during the freeze-drying step has advantageous effects on the quality of the matrixes A and B. According to the invention, the cooling rate is up to 50° C. per hour, more particularly 5° C. to 40° C. per hour, advantageously 10° C. to 30° C. per hour, preferably 15° C. to 25° C. per hour, more preferably 18° C. to 23° C. per hour and even more preferably 20° C. to 22° C. per hour.

Such preferably obtainable lyophilizates have a small skin on the product surface.

This small skin advantageously forms pores in the surface of the freeze-dried matrix which offer particularly good conditions for the subsequent population with fibroblasts because they promote slow migration of the fibroblasts into the matrix. By contrast, faster cooling rates during the freeze-drying step often lead to an open-pored matrix with “craters” in the surface which the fibroblasts are unable completely to fill with newly synthesized extracellular matrix in the given cultivation time. Sown keratinocytes are able to drop into these craters and to aggregate in, and diffuse from, them so that the layering and hence the differentiation of the skin model can be significantly affected.

The cross-linking step d) can be carried out by any physical or chemical cross-linking process that appears suitable to the expert. Applicants have surprisingly found that, despite its cytotoxic and apoptotic properties, glutaraldehyde is a particularly suitable cross-linking agent for the process according to the invention. However, cross-linking can also be carried out by physical processes, such as UV irradiation and dehydrothermal cross-linking (DHT).

According to the invention, suitable chemical cross-linking agents are bifunctional substances of which the groups react with the amino groups of the lysine and hydroxylysine residues on various polypeptide chains of the collagen fibers. In addition, activation of the carboxyl groups of the free glutamic and aspartic acid residues, followed by reaction with the amino groups of another polypeptide chain, can lead to a cross-linking according to the invention of the collagen fibers. According to the invention, cross-linking between two collagen fibers can also be achieved by reaction of the amino groups with diisocyanates or by the formation of acyl azides. Another standard cross-linking method which may be used for the purposes of the invention is cross-linking with carbodiimides, such as EDC (1-ethyl-3-(3-dimethylaminopropyl)-carbodiimide) for example, in conjunction with N-hydroxysuccinimide. According to the invention, cross-linking reactions with homo/bifunctional cross-linking agents reacting with amino groups, for example, with p-benzoquinone, dimethyl adipimidate, dimethyl pimelinidate, dimethyl suberimidate, 1,4-phenylene diisothiocyanate, polyoxyethylene-bis-(imidazolylcarbonyl), bis-[polyoxyethylene-bis-(imidazolylcarbonyl)] and suberic acid-bis-(N-hydroxysuccinimide ester), are also possible, as is the use of a biological cross-linking agent, more particularly with enzymes, preferably transglutaminase, which is capable of linking peptide chains to one another, or lysyl oxidases (EC 1.4.3.13). Other cross-linking options are mentioned in EP-A2-0898973 to which reference is made here.

The fibroblasts and keratinocytes are obtained and cultivated by methods known among experts which may be adapted to the required properties of the skin model to be produced.

In addition, in a preferred embodiment of the invention, other cell types and/or other cells of other tissue types of both human and animal origin, for example, of mammals, and/or precursor cells thereof, for example, melanocytes, macrophages, monocytes, leukocytes, plasma cells, neuronal cells, adipocytes, induced and non-induced precursor cells of Langerhans cells, Langerhans cells and other immune cells, endothelial cells, cells from tumors of the skin or skin-associated cells, more particularly sebocytes or sebaceous gland tissue or sebaceous gland explantates, cells of the sweat glands or sweat gland tissue or sweat gland explantates, hair follicle cells or hair follicle explantates; and cells from tumors of other organs or from metastases, may be sown on the matrix before, during or after sowing of the keratinocytes in step f). Stem cells of various origins, tissue-specific stem cells, embryonal and/or adult stem cells may also be incorporated in the skin model. Accordingly, the process according to the invention provides an organoid in-vitro skin model which is made up of two tissue-specific layers, namely a dermis equivalent and an epidermis equivalent. The organotypical skin model substantially corresponds to native skin both histologically and functionally.

The effect of the cross-linking of matrix A is that the dermis equivalent growing on and in matrix B undergoes very little or no shrinkage during the period of cultivation. Skin equivalents with a defined diameter, a uniform surface and a defined border with the rim of the culture vessel are thus obtained. By virtue of the uniform size and uniform quality of the whole skin model used as a test surface, the quality of the results in the testing of substances for pharmacological and/or cosmetic effects is increased and the test results become more reproducible.

Accordingly, the matrix B intended for cultivation of the fibroblasts contains the fibroblasts to be cultivated and a collagen framework newly constituted from a preferably fresh collagen suspension of human or animal origin in a concentration of about 5 to 50 mg collagen per ml matrix, corresponding to 0.5% to 5% collagen. The range from 8 to 25, corresponding to 0.8% to 2.5% collagen, is preferred and the range from 8 to 12 mg collagen per ml matrix, corresponding to 0.8% to 1.2% collagen, particularly preferred.

The collagen framework is obtained from a preferably cell-free acidic suspension of poorly soluble collagen, the protein concentration of the suspension preferably being from 5 to 15 mg/ml, corresponding to 0.5 to 1.5% by weight. The pH value of the collagen solution is in the range from 0.1 to 6.9, preferably in the range from 2.0 to 5.0, more preferably in the range from 3.0 to 4.5 and most preferably in the range from 3.5 to 4.0.

To cultivate the fibroblast-containing matrix, a solution containing a cell culture medium (preferably DMEM cell culture medium), buffer (for example, Hepes buffer), serum (preferably fetal calf serum (FCS), normal calf serum (NCS), normal lamb serum (NLS), defined serum or serum substitutes) and preferably 1-6×10⁵ matrix fibroblasts, more particularly precultivated fibroblasts, is added to the matrix B.

In order to further stabilize the matrix B, fibronectin and/or laminin, preferably human fibronectin and/or laminin, may be added onto the matrix or into the culture medium. Fibronectins are structural or adhesion proteins produced in fibroblasts of which the function in vivo is to bind to other macromolecules, for example, collagen, and to attach cells to neighboring cells. Laminin is a protein of the basal membrane to which cells are able to adhere. Accordingly, the addition of fibronectins and/or laminin to the fibroblast-collagen matrix promotes the binding of the fibroblasts both to collagen and to one another. Both proteins may either be directly incorporated in the matrix during its production or may be added to the matrix in dissolved form, for example, dissolved in culture medium, after the cross-linking step. This may be done before or parallel to the cell sowing step.

The subsequent cultivation of the fibroblasts in the collagen matrix preferably takes place by submerse culture. In the context of the present invention, “submerse cultivation” or “submerse culture” is understood to be a process for cultivating cells, the cells being covered with a nutrient solution. Accordingly, the fibroblast-containing matrix B is preferably submerged in cell culture medium and incubated at temperatures of about 30° C. to about 40° C.

In addition to the described method for cultivating a whole skin model obtained in accordance with the invention, the model can be optimized by changes to the cultivation conditions, for example, by medium components, to the extent that it acquires a better barrier function which comes even closer to the in vivo situation. This is important, for example, for carrying out penetration studies and also for the production of products which improve the barrier function of the skin. In addition, there are medically relevant disturbances to the barrier function either through environmental factors (contact with detergents) or genetic causes. Skin models with an optimized barrier function would be important here. The improvement in the barrier function can be achieved by modified culture conditions (for example, atmospheric moisture, temperature), by chemical components in the medium (ceramides, vitamin C) or by genetically modified keratinocytes. The change in the barrier function can be measured by measuring the surface electrical capacitance. Detailed information on improving the barrier function can be found, for example, in Published United States Patent Application No. US-A1-200220168768, to the full disclosure of which reference is made here.

In order to improve fibroblast growth in the dermis equivalent, factors may be added to the culture medium which, in the case of co-cultivation of fibroblasts and keratinocytes, are released by the keratinocytes. For example, the conditioned culture supernatant (i.e. the medium supernatant of cultivated keratinocytes and/or co-cultures of keratinocytes with other cell types, such as endothelial cells, immune cells or fibroblasts, for example) may be used.

In another advantageous embodiment of the invention, the fibroblasts are cultivated in the matrix B, as described above, in such a way that a dermis equivalent can be subsequently obtained. This is preferably done by a process for the production of a dermis equivalent comprising the steps of: (a) providing a poorly soluble collagen obtained from collagen-containing tissue; (b) forming a homogeneous aqueous suspension by mixing the collagen with aqueous medium; (c) forming a first matrix (A) by lyophilizing the collagen suspension; (d) forming a second matrix (B) by cross-linking the collagen in the first matrix (A) to form a mechanically stabilized second matrix (B); (e) sowing fibroblasts onto the second matrix (B) and allowing the fibroblasts to grow; (f) further cultivating the fibroblasts growing in and on matrix (B) to form a dermis equivalent.

In the context of the present invention, a “dermis equivalent” is understood to be a connective-tissue-like layer of collagen and fibroblasts which substantially corresponds to the native dermis.

The dermis equivalent thus obtained may be used for screening and diagnosis processes, more particularly for studying the effects of chemical substances, plant extracts and metals, for example, potential medicaments or constituents of cosmetics, or other agents (physical quantities), such as light or heat, radioactivity, sound, electromagnetic radiation, electrical fields and also for studying phototoxicity, i.e. the damaging effect of light of different wavelengths on cell structures. The dermis equivalent produced in accordance with the invention may also be used for studying wound healing and is also suitable for studying the effects of gases, aerosols, smoke and dusts on cell structures or the metabolism or gene expression.

In the context of the present invention, an “agent” or “agents” is/are understood to be physical media, such as light, heat or the like acting on the skin or skin cells. Accordingly, the present invention also relates to screening and diagnosis processes using the dermis equivalents produced in accordance with the invention.

A preferred embodiment of the invention comprises the treatment of the dermis equivalent in the presence and absence of the substance to be studied and/or the agent to be studied and comparison of the effects observed on the cells or cell constituents of the dermis equivalent.

Another preferred embodiment of the invention is a process for studying the penetration of substances using the dermis equivalent produced in accordance with the invention and using a whole skin equivalent according to the invention consisting of a dermis equivalent and an epidermis equivalent.

In another particularly preferred embodiment, the present invention also relates to a process of the type mentioned above for cultivating dermal fibroblasts and epidermal keratinocytes in a matrix for the production of a whole skin equivalent consisting of dermis equivalent and epidermis equivalent. In this process, keratinocytes are sown on the matrix one to three weeks and preferably 10 to 14 days after the above-described production and incubation of the fibroblast-containing collagen matrix.

In the context of the present invention, “keratinocytes” are understood to be cells of the epidermis which form keratinizing plate epithelium, genetically modified keratinocytes or keratinocytes emanating from spontaneous mutations or precursors of such keratinocytes which may be of animal or human origin. Since the development of a well-differentiated epidermis with intact keratinization depends in large measure on the proportion of basal stem cells in the keratinocytes used, the keratinocytes sown on the matrix in step f) of the process according to the invention may be largely undifferentiated keratinocyte stem cells from human biopsy tissue. However, cell lines or certain differentiated cells may also be used. Alternatively to the normal skin keratinocytes, mucous membrane keratinocytes or intestinal epithelial cells may be applied to the matrix. These are preferably precultivated cells and, in a particularly preferred embodiment, keratinocytes in the first or in the second cell passage, although cells from higher passages may also be used.

The sowing of the keratinocytes on the matrix preferably takes place in a cell culture medium and, in a particularly preferred embodiment, in DMEM/F12 medium which contains approximately 1 to 30% fetal calf serum, NCS, defined serum or serum substitute products and—in varying concentrations—various additives which promote the proliferation and differentiation of the cells. The matrix is then preferably submerged in DMEM medium containing, in particular, EGF from mice or even comparable preparations from other animals, epidermal growth factor (hEGF) (for example, in a concentration of 0.2 μg/1 medium) and, for example, 0.8 mM CaCl₂ and subjected to submerse cultivation for preferably 1 to 10 days and more particularly 3 to 7 days.

Complete differentiation of the keratinocyte layers is achieved, for example, by an airlift culture in DMEM without hEGF and BPE (bovine pituitary extract). In the context of the present invention, an “airlift culture” is understood to be a culture where the height of the nutrient medium level is adapted exactly to the height of the matrix whereas the keratinocytes or cell layers formed by the keratinocytes lie above the nutrient medium level and are not covered by the nutrient medium, i.e. cultivation takes place at the air/nutrient medium interface, the cultures being fed from below. To this end, the skin models, for example, are lifted from a microtiter plate and placed on filter papers resting on metal spacers in Petri dishes. The medium is introduced into the Petri dishes to such a level that it does not completely cover the filter paper, instead a liquid collar is present around the base of the skin models (air/liquid interface). The duration of the airlift culture may be varied as required by the expert. Typically, it is about 1 to 4 weeks. During this period, a skin-typical in-vitro whole skin model consisting of dermis equivalent and epidermis equivalent is developed.

The process according to the invention for the production of an in-vitro whole skin model may advantageously be modified so that other cell types, such as melanocytes, macrophages, monocytes, leucosytes, plasma cells, neuronal cells, adipocytes, induced and non-induced precursor cells of Langerhans cells, Langerhans cells and other immune cells, endothelial cells and cells from tumors of the skin or skin-associated cells, can be sown on the matrix and further cultivated before, during or after sowing of the keratinocytes. The cells mentioned may be of human and animal origin.

Accordingly, the present invention also relates to a skin-typical in vitro whole skin model, more particularly a human in-vitro whole skin model, which has been produced by the process according to the invention and an optionally following and/or preceding conventional cultivation process and which, in the epidermal part, comprises at least one proliferative cell layer, a few differentiating cell layers and at least one keratinzed cell layer, the epidermis equivalent comprising Stratum basale, Stratum spinosum, Stratum granulosum and Stratum corneum and a basal membrane consisting of the characteristic BM proteins, such as laminin and collagen IV, for example, being present between the dermis equivalent and the epidermis equivalent and, in addition, skin-typical proteins, such as transglutaminase, involucrin, collagen IV, laminin, filaggrin, fibronectin, K-67, cytokeratin 10 and, in particular, elastin being expressed.

However, the stress marker cytokeratin 6 is only expressed to a minimal extent which indicates that the cells in the in-vitro whole skin model according to the invention are in a relatively low-stress state. Strong CK6 expression is observed in the skin, for example, during wound healing or regeneration.

The proliferative behavior and the differentiation of the dermal and epidermal cells can be modified by applying electrical or electromagnetic fields to the dermis model or whole skin model during the cultivation phase.

In view of its complexity, the whole skin model produced can be specifically used for tackling various problems in the chemical/pharmaceutical industry and in the cosmetics industry. More particularly, the skin equivalent produced in accordance with the invention is suitable for testing products, for example, for effectiveness, unwanted side effects, for example, irritation, toxicity and inflammation or allergenic effects, or the compatibility of substances. These substances may be substances intended for potential use as medicaments, more particularly as dermatics, or substances which are constituents of cosmetics or even consumer goods which come into contact with the skin, such as, for example, laundry detergents, etc.

The skin equivalent produced in accordance with the invention may also be used, for example, for studying the absorption, transport and/or penetration of substances. It is also suitable for studying other agents (physical quantities), such as light or heat, radioactivity, sound, electromagnetic radiation, electrical fields, for example, for studying phototoxicity, i.e. the damaging effect of light of different wavelengths on cell structures. The skin equivalent produced in accordance with the invention may also be used for studying wound healing and is also suitable for studying the effects of gases, aerosols, smoke and dusts on cell structures or the metabolism or gene expression.

The effects of substances or agents on human skin can be determined, for example, from the release of substances, for example, cytokines or mediators, by cells of the human or animal skin model system and the effects on gene expression, metabolism, proliferation, differentiation and reorganization of those cells. Using processes for quantifying cell damage, more particularly using a vital dye, such as a tetrazolium derivative, it is possible, for example, to detect cytotoxic effects on skin cells. The testing of substances or agents using the human skin equivalent according to the invention may comprise both histological processes and also immunological and/or molecular-biological processes.

Accordingly, a preferred embodiment of the invention comprises processes for studying the effect, more particularly pharmacological effects, of substances or agents on human skin using the human skin equivalent produced in accordance with the invention. In a particularly preferred embodiment, an XTT tetrazolium reduction test (EZ4U test) or Alamar Blue (resazurin reduction test, Promega) is carried out. EZ4U is a non-toxic water-soluble yellow tetrazolium salt which can be reduced by living cells to intensively colored formazanes. The reduction requires intact mitochondria so that the test can be used to assess the vitality of cells. Vitality assays usable in accordance with the invention, which are also based on tetrazolium compounds, are, for example, the MTT, MTS or WST-1 test. These are available as test kits inter alia from Roche. Cell vitality can also be assessed from the release of lactate dehydrogenase (LDH) from cells with damaged cell membrane. Cells with damaged cell membrane can be selectively colored with Trypan Blue.

Another preferred embodiment of the invention comprises a process for studying the penetration of substances in which both a dermis equivalent produced in accordance with the invention and a skin equivalent produced in accordance with the invention are treated with the substances to be studied and the results obtained with both systems are compared with one another.

In another particularly preferred embodiment of the invention, the effects of chemical substances or other agents on special skin types are investigated. In this embodiment, cells of defined skin types, for example, skin types with few pigments and/or skin types with numerous pigments, are used to establish skin equivalents according to the invention which are tested for the effect of substances or agents.

In another particularly preferred embodiment of the invention, the skin equivalent produced in accordance with the invention is used as a model system for studying skin diseases and for the development of new treatments for skin diseases. For example, cells of patients with a certain genetic or acquired skin disease may be used to establish patient-specific skin model systems which may in turn be used to study and evaluate the effectiveness of certain therapies and/or medicaments.

In a preferred embodiment, the skin equivalent produced in accordance with the invention may be populated with microorganisms, more particularly pathogenic microorganisms. Population with pathogenic or parasitic microorganisms, including, in particular, human-pathogenic microorganisms, is particularly preferred. Microorganisms in the context of the present invention are, in particular, fungi, bacteria and viruses. The microorganisms are preferably selected from fungi or pathogenic and/or parasitic bacteria. Particularly preferred fungi are species of the genus Candida albicans, Trichophyton mentagrophytes and Malassezia furfur. Particularly preferred pathogenic and/or parasitic bacteria are Staphylococcus aureus.

Using a correspondingly populated skin equivalent, it is possible to study both the process of population, more particularly the infection process, by the microorganism itself and the response of the skin to that population. In addition, the effect of substances applied before, during or after the population on the population itself or on the effects of the population on the skin equivalent can be studied with a skin equivalent of the type in question.

The present invention also relates to a cross-linked matrix in which the above-mentioned cultivation process can be carried out. As described above, the combination according to the invention of matrix and fibroblasts cultivated therein may be used for the production of a dermis equivalent, an epidermis equivalent and/or an organoid whole skin model.

The present invention also relates to a process for the production of an epidermis model comprising the steps of: (a) providing a poorly soluble collagen obtained from collagen-containing tissue; (b) forming a homogeneous aqueous suspension by mixing the collagen with aqueous medium; (c) forming a first matrix (A) by lyophilizing the collagen suspension; (d) forming a second matrix (B) by cross-linking the collagen in the first matrix (A) to form a mechanically stabilized second matrix (B); (e) sowing keratinocytes onto the second matrix B and allowing the keratinocytes to grow; (f) further cultivating the keratinocytes on matrix (B) to form an epidermis equivalent.

In this case, in contrast to the process according to the invention for the production of the whole skin model—only keratinocytes which act as a framework or carrier for the epidermis model are sown on the matrix and cultivated under such conditions that the keratinocytes first proliferate and then differentiate themselves so that an epidermis consisting of all four layers is formed. The matrix is preferably pretreated with proteins of the extracellular matrix or the basal membrane in order to achieve better adhesion of the keratinocytes to the matrix material and to offer the cells signals provided by these proteins. Examples of proteins suitable for the purposes of the invention are collagen IV or collagens of other types, fibronectin and laminin. The present invention also relates to the epidermis model thus formed which, like the whole skin model, may also be used for diverse tests.

Preferred culture mediums are DMEM (Dulbecco's Modified Eagle Medium), M199 and Ham's F12 Medium. However, any other cell culture medium which allows the cultivation of fibroblasts may also be used. Fetal calf serum (FCS) is preferably used as the serum, although NCS and serum substitute products are also suitable, while Hepes buffer, for example, is used as the buffer. The pH value of the solution of cell culture medium, buffer and serum is preferably in the range from 6.0 to 8.0, for example, from 6.5 to 7.5 and, more particularly, 7.0.

According to the invention, the medium may contain other factors, for example, hormones, growth factors, adhesion proteins, antibiotics, selection factors, enzymes and enzyme inhibitors and the like.

To improve keratinocyte growth in the epidermis equivalent, factors may be added to the culture medium which—in the case of co-cultivation of fibroblasts and keratinocytes—are released by the fibroblasts. For example, the conditioned culture supernatant (i.e. the medium supernatant of cultivated fibroblasts and/or co-cultures of fibroblasts with other cell types, such as, for example, endothelial cells, immune cells or keratinocytes) may be used.

The following Figures and Examples are intended to illustrate the invention without limiting it in any way.

### EXAMPLE 1

Production of Matrix A.

700 ml deionized water acidified with 875 μl concentrated acetic acid (glacial acetic acid) are poured into a 1,000 ml glass beaker. The solution is stirred with a commercially available magnetic stirrer comprising a stirring fish. 7.0 g bovine tendon collagen are stirred all at once into the rotating water column, after which the mixture is taken from the stirrer and left standing for 4 hours at room temperature without stirring. The pH of the mixture is monitored every 30 minutes and should be in the range from 3.5 to 4. The mixture is stirred at high speed for 1 minute every 30 minutes. It is important to ensure that the temperature of the suspension does not rise. Finally, the suspension is stirred at high speed for 3 minutes and thus homogenized. In order to avoid heating by the homogenization process, the suspension is kept at room temperature. A milky collagen suspension is obtained. In order to remove any air bubbles present, the suspension may be centrifuged at 400 r.p.m. (=24.73 G) for 1 minute at 20° C. (Hettich Universal 16 R, rotor diameter 138 mm). The bubbles then escape without foam generation. Using a dispenser, the suspension is pipetted into the wells of a 24-well cell culture plate. The filled plate is then taken up by hand, tilted slightly and turned in all directions so that the gel solution wets the wall of the wells even above the matrix surface. This improves the adhesion of the matrixes in the wells. The filled plates thus treated are directly placed in the freeze dryer and frozen therein.

The collagen suspension introduced into the 24-well cell culture plates is frozen in a freeze dryer with heatable shelves and then dried under reduced pressure.

Starting from a temperature of the gel solution of 20° C., the freezing rate is 18° C. to 23° C. per hour. The entire freeze drying process lasts approximately 20 to 27 hours.

### EXAMPLE 2

Cross-Linking of Matrix A to Produce Matrix B.

In order to obtain skin models with a large and flat surface, the collagen matrixes are chemically fixed before sowing with the fibroblasts. Glutaraldehyde (GA) is used as the fixing agent: C₅H₈O₂, MW=100.12.

Glutaraldehyde solution is carefully pipetted onto each matrix of 24 matrixes A in a 24-well cell culture plate. The solution is allowed to run slowly down the inner rim of the well in order not to damage the matrix surface. The glutaraldehyde solution takes several minutes to penetrate into the interior of the collagen sponges. The increasing saturation is reflected in a change in the color of the matrixes from pure white to gray—pale yellow. The cover is then placed on the cell culture plates and the edge is hermetically sealed with Parafilm to avoid evaporation of the solution. The treated plates are stored for 24 hours at room temperature protected from light.

The cross-linking of the matrixes, including washing and equilibration, can be carried out over a period of preferably 5 days, but at least 4 days.

### EXAMPLE 3

Production of a Dermis Equivalent.

Before sowing onto the cross-linked collagen matrixes, fibroblasts of a suitable passage are precultivated in cell culture bottles containing fibroblast medium. After the required cell density has been reached, the culture medium is removed under suction. The cells are detached from the bottom of the culture bottles by addition of a trypsin solution, washed with culture medium and removed by centrifuging. After determination of the cell count, the cell suspension is adjusted to a concentration of 1-6×10⁵ fibroblasts/ml. The medium is sucked from the wells of the microtiter plate, which contain the matrixes equilibrated with fibroblast medium, to such an extent that the matrixes remain moist. Quantities of 1 ml fibroblast medium each containing 1-6×10⁵ fibroblasts are pipetted onto the surface of the matrixes without damaging the surface. On completion of sowing, the cover is placed on the plate which is then placed horizontally in the incubator. The cultivation of the fibroblasts on the matrix takes place at 37° C./5% v/v CO₂. The culture medium is changed at regular intervals. After 2-4 weeks, the dermis model is fully developed.

### EXAMPLE 4

Production of a Whole Skin Model.

Before sowing onto the cross-linked collagen matrixes, fibroblasts of a suitable passage are precultivated in cell culture bottles containing fibroblast medium. After the required cell density has been reached, the culture medium is removed under suction. The cells are detached from the bottom of the culture bottles by addition of a trypsin solution (or any other solution suitable for detaching adhering cells), washed with fibroblast medium and removed by centrifuging. After determination of the cell count, the cell suspension is adjusted to a concentration of 1-6×10⁵ fibroblasts/ml. The medium is sucked from the wells of the microtiter plate, which contain the matrixes equilibrated with culture medium, to such an extent that the surface of the matrixes remain moist. Quantities of 1 ml fibroblast medium each containing 1-6×10⁵ fibroblasts are pipetted onto the surface of the matrixes without damaging the surface. On completion of sowing, the cover is placed on the plate which is then placed horizontally in the incubator. The cultivation of the fibroblasts on the matrix takes place at 37° C./5% v/v CO₂. The culture medium is changed at regular intervals. After a fibroblast cultivation period of 2-4 weeks, the keratinocytes are sown onto the dermis model. To this end, keratinocytes of a suitable passage are precultivated in cell culture bottles containing keratinocyte medium. After the required cell density has been reached, the culture medium is removed under suction. The cells are detached from the bottom of the culture bottles by addition of a trypsin solution, washed with culture medium (keratinocyte medium) and removed by centrifuging. After determination of the cell count, the cell suspension is adjusted to a concentration of 1-6×10⁵ keratinocytes/ml. The medium is sucked from the wells of the microtiter plate, which contain the dermis equivalents, to such an extent that the surface of the dermis equivalents remain moist. Quantities of 1 ml keratinocyte medium each containing 1-6×10⁵ keratinocytes are pipetted onto the surface of the matrixes without damaging the surface. On completion of sowing, the cover is placed on the plate which is then placed horizontally in the incubator (submerse culture). The cultivation of the fibroblasts and keratinocytes on the matrix takes place for 3 to 7 days at 37° C./5% v/v CO₂. The culture medium is changed at regular intervals.

After the 3 to 7 day submerse culture, the keratinocyte medium is removed from the wells under suction. The still incomplete whole skin models are removed from the wells and placed on filter papers. The filter papers lie on metal spacers in a Petri dish. After the incomplete whole skin models have been placed on the filter paper, the Petri dish is filled with culture medium (airlift medium, air/liquid interface medium) to such an extent that the medium reaches the upper edge of the filter paper and spreads around the base of the skin models. The surface of the skin models is not covered with culture medium (airlift culture or air/liquid interface). The skin models are left in the air/liquid interface for 1 to 4 weeks according to the required degree of differentiation. The culture medium is changed at regular intervals.

The composition of the three different culture mediums is shown in the following Tables.

Fibroblasts.

| | Final concentration | |
|---|---|---|
| DMEM (Glutamax I) | | |
| FCS | 10% | |
| Penicillin G | 100 | Ul/ml |
| Gentamicin | 25 | μg/ml |
| Ascorbyl-2-phosphate | 1 | mM |

Keratinocytes.

| | Final concentration | |
|---|---|---|
| DMEM (Glutamax I) | | |
| HAM F 12 | | |
| Fetal Clone II | 10% | |
| EGF | 10 | ng/ml |
| Hydrocortisone | 0.4 | μg/ml |
| Insulin | 0.12 | Ul/ml |
| Choleratoxin | 10⁻¹⁰ | M |
| Tri-iodothyrionine | 2*10⁻⁹ M (5 μg/ml) | |
| Adenine | 2.43 | μg/ml |
| Penicillin G | 100 | Ul/ml |
| Gentamicin | 25 | μg/ml |
| Ascorbyl-2-phosphate | 1 | mM |

Air/liquid interface.

| | Final concentration | |
|---|---|---|
| DMEM (Glutamax I) | | |
| HAM F 12 | | |
| Hydrocortisone | 0.4 | μg/ml |
| Insulin | 0.12 | Ul/ml |
| Penicillin G | 100 | Ul/ml |
| Gentamicin | 25 | μg/ml |
| BSA | 1.6 | mg/ml |
| Ascorbyl-2-phosphate | 1 | mM |

### EXAMPLE 5

Detection of Elastin in the Whole Skin Model.

Ready-differentiated skin models are directly removed from the air/liquid interface, frozen in a cryostat and cut (thickness 8 μm). The sections are fixed for 10 minutes in −20° C. cold acetone and then repeatedly washed with TBS (TRIS-buffered saline). The anti-elastin antibody (rabbit: Novotec) is diluted 1:40 with TBS, added dropwise to the sections and left on them for 60 minutes. The sections are then washed three times for 5 minutes in TBS. A goat/anti-rabbit antibody diluted 1:20 with Alexa conjugate (from Molecular Probes) is used as a secondary antibody. In order to block the high natural fluorescence of the collagen matrix, the secondary antibody is diluted in a 0.1% Evans Blue solution. The secondary antibody is added dropwise to the sections and left on them for 60 minutes. They are then washed three times for 5 minutes with TBS. Finally, the antibody-marked sections are coverslipped in DAKO Faramount embedding medium. Sections without the first antibody are incubated as a negative control.

1. A process for the production of a whole skin model comprising the steps of: (a) providing a poorly soluble collagen obtained from collagen-containing tissue; (b) forming a homogeneous aqueous suspension by mixing the collagen with an aqueous medium; (c) forming a first matrix (A) by lyophilizing the collagen suspension; (d) forming a second matrix (B) by cross-linking the collagen in the first matrix (A) to form a mechanically stabilized second matrix (B); (e) sowing fibroblasts onto the second matrix (B) and allowing the fibroblasts to grow; (f) sowing keratinocytes onto the second matrix B and allowing the keratinocytes to grow; (g) further cultivating the fibroblasts and keratinocytes growing in and on matrix (B) to form a complete whole skin model comprised of a dermal and epidermal part.

2. A process for the production of a collagen matrix comprising the steps of: (a) providing a poorly soluble collagen obtained from collagen-containing tissue; (b) forming a homogeneous aqueous suspension by mixing the collagen with aqueous medium; (c) forming a first matrix (A) by lyophilizing the collagen suspension; (d) forming a second matrix (B) by cross-linking the collagen in the first matrix (A) to form a mechanically stabilized second matrix (B).

3. A process for the production of a dermis equivalent comprising the steps of: (a) providing a poorly soluble collagen obtained from collagen-containing tissue; (b) forming a homogeneous aqueous suspension by mixing the collagen with aqueous medium; (c) forming a first matrix (A) by lyophilizing the collagen suspension; (d) forming a second matrix (B) by cross-linking the collagen in the first matrix (A) to form a mechanically stabilized second matrix (B); (e) sowing fibroblasts onto the second matrix (B) and allowing the fibroblasts to grow; (f) further cultivating the fibroblasts growing in and on matrix (B) to form a dermis equivalent.

4. A process for the production of an epidermis equivalent comprising the steps of: (a) providing a poorly soluble collagen obtained from collagen-containing tissue; (b) forming a homogeneous aqueous suspension by mixing the collagen with aqueous medium; (c) forming a first matrix (A) by lyophilizing the collagen suspension; (d) forming a second matrix (B) by cross-linking the collagen in the first matrix (A) to form a mechanically stabilized second matrix (B); (e) sowing keratinocytes onto the second matrix B and allowing the keratinocytes to grow; (f) further cultivating the keratinocytes on matrix (B) to form an epidermis equivalent.

5. The process of claim 1 wherein the poorly soluble collagen is a coarse-fiber collagen which shows no visible swelling or gelling or only slight swelling or gelling after several hours in aqueous medium.

6. The process of claim 5 wherein the poorly soluble collagen is obtained from the tendons of horses, pigs or cattle.

7. The process of claim 1 wherein the lyophilization step (c) is carried out at a cooling rate of up to 50° C. per hour.

8. The process of claim 7 wherein the cooling rate is 20° C. to 22° C. per hour.

9. A whole skin model produced by the process of claim 1.

10. The whole skin model of claim 9 wherein elastin is expressed.

11. The whole skin model of claim 9 populated with a microorganism.

12. The whole skin model of claim 11 wherein the microorganism is selected from the group consisting of Staphylococcus aureus and species of the genus Candida, Malassezia and Trichophyton.

13. A collagen matrix produced by the process of claim 2.

14. A dermis equivalent produced by the process of claim 3.

15. An epidermis equivalent produced by the process of claim 4.
